(19) United States
(12) Patent Application Publication
(10) Pub. No.: US 2025/0255524 A1
(43) Pub. Date: Aug. 14, 2025

(54) DEVICES AND SYSTEMS FOR AUTOMATED COLLECTION OF BLOOD INTO TUBE STORED AT ATMOSPHERIC PRESSURE AND MIXING OF THE BLOOD WITH ADDITIVES IN THE TUBE

(71) Applicant: P5 Designers, LLC., Milford, NJ (US)

(72) Inventors: Paul D. CARSE, Milford, NJ (US); Cordelia A. OGREN, Milford, NJ (US); Kelly Gail DUNCAN, Bethlehem, PA (US); William CARR, Phillipsburg, NJ (US); David TAVOR, Hod Hasharon (IL); Rachel Jessica BALJI, Mahwah, NJ (US)

(21) Appl. No.: 18/996,654

(22) PCT Filed: Jun. 24, 2024
(86) PCT No.: PCT/IL2024/050615

§ 371 Date: Jan. 17, 2025

Related U.S. Application Data
(60) Provisional application No. 63/510,380, filed on Jun. 27, 2023.

(51) Int. Cl. A61B 5/15

(52) U.S. Cl. CPC A61B 5/150946; A61B 5/15003; A61B 5/150099; A61B 5/150213; A61B 5/150267; A61B 5/150335; A61B 5/150755; A61B 5/150763

(57) ABSTRACT

A blood collection tube comprises: 1) a body including a proximal end and a distal end, and defining an axial extent between the proximal end and the distal end; 2) a chamber within the body, the chamber including a plurality of mixing ribs; 3) a vacuum port; and 4) a cap arranged on the proximal end, wherein the cap comprises: a) a top face including a first septum; b) a first conduit having an inlet extending from the first septum and an outlet in fluid communication with the chamber; c) a lateral face including a second septum; d) a second conduit extending radially from the second septum to the first conduit; and a plurality of mixing ribs; wherein, when a fluid source is fluidically connected to the first septum or the second septum, and a vacuum is applied at the vacuum port, the vacuum draws fluid from the fluid source, through the first or second septum, and into the first conduit; and when the tube is rotated around an axis defined by the axial extent, the mixing ribs of the cap and the mixing ribs of the chamber cause fluids therein to swirl, thereby effecting mixing of the fluids therein.

### FIELD OF THE INVENTION

The present Application relates to the collection of blood into blood collection tubes for testing and, more specifically, but not exclusively, to a blood collection tube, blood collection device, and method for collecting blood into tubes stored at atmospheric pressure.

### BACKGROUND OF THE INVENTION

Analysis of blood samples is one of the most important diagnostic tools available for detecting and treating diseases. Blood samples are obtained by a process known as venipuncture. During venipuncture, a cannula is inserted into a vein, and blood is drawn through tubing attached to the cannula into one or more blood collection tubes. The blood collection tubes may have one or more additives to prevent coagulation or isolate specific blood components.

For over 70 years, the industry standard for blood collection tubes has been the evacuated blood collection tube, such as the Vacutainer® manufactured by Becton Dickinson. An evacuated blood collection tube is prepared by adding a blood additive into the tube, stopping the tube, applying a vacuum to the tube, and storing the tube in internal vacuum conditions. Evacuated blood collection tubes account for nearly 1% of global medical supply sales, and it is estimated that annual global sales of evacuated blood collection tubes exceed $1.7 billion.

International Patent Publication WO2022/038616, entitled “Devices and Systems for Automated Collection of Blood into Tube Stored at Atmospheric Pressure,” and assigned to the same assignee as the present disclosure, discloses a blood collection system that is designed to withdraw blood at micro-vacuum, to tubes that are not evacuated. The contents of that application are incorporated by reference as if fully set forth herein.

### SUMMARY OF THE INVENTION

The present disclosure builds upon the disclosure of the aforementioned International Patent Publication WO2022/038616 to provide additional embodiments of blood collection systems that are designed to withdraw blood at micro-vacuum. A blood collection tube has a first septum for the input of blood, a second septum for the input of a blood additive, and a vacuum port. A blood collection device includes a vacuum pump and a plurality of reservoirs containing blood additives. When the blood collection tube is inserted into the blood collection device, the vacuum pump applies a vacuum through the vacuum port. The applied vacuum draws into the blood collection tube both an aliquot of the concentrated blood additive and a predefined blood volume. Optionally, the additive may be added by other means, such as an injector. The blood additive and blood are then mixed in the blood collection tube. Prior to the collection, the phlebotomist confirms the patient's identity, labels the collection tubes, and orders the blood collection tubes, e.g., with a scanner built into the blood collection device. The blood collection is performed at a low vacuum and draws the minimum required quantity of blood, with virtually no risk of misidentification of patients, incorrect ordering of collection tubes, or mislabeling of specimens.

In particular, the blood collection tube and the cap include mixing ribs. The mixing ribs are designed to induce the mixing of the blood and the additive when the tube is rotated. Furthermore, as an alternative to adhesive paper labeling mentioned previously, the blood collection device may include a laser for etching, and the tube is made of a material susceptible to laser etching (such as polycarbonate). The tube may also include sizing (e.g., a glaze or surface coating) to increase contrast during the printing. Optionally, the sizing material may also serve to protect the contents of the tube during the etching process. During the rotation of the tube for mixing, a laser is applied to the tube to imprint patient identification. Advantageously, mixing through rotation, with the operation of the mixing ribs, provides thorough mixing between the blood and the additive, which is especially necessary when small volumes of the blood and additive are employed. Furthermore, the laser etching process is quick, reliable, automated, and uses less consumable materials than printed labels. The laser etching process may be synchronized with the mixing process, enabling timely completion of the blood collection process.

According to a first aspect, a blood collection tube is disclosed. The blood collection tube includes a body including a proximal end and a distal end, and defining an axial extent between the proximal end and the distal end; a chamber within the body, the chamber including a plurality of mixing ribs; a vacuum port; and a cap arranged on the proximal end. The cap includes a top face including a first septum; a first conduit having an inlet extending from the first septum and an outlet in fluid communication with the chamber; a lateral face including a second septum; a second conduit extending radially from the second septum to the first conduit; and a plurality of mixing ribs. When a fluid source is fluidically connected to the first septum or the second septum, and a vacuum is applied at the vacuum port, the vacuum draws fluid from the fluid source through the first or second septum, and into the first conduit, and when the tube is rotated around an axis defined by the axial extent, the mixing ribs of the cap and the mixing ribs of the chamber cause fluids therein to swirl, thereby effecting mixing of the fluids therein.

In another implementation, according to the first aspect, the tube includes a relief channel configured to enable air to escape from the chamber during blood filling.

In another implementation according to the first aspect, a system for collecting blood includes the blood collection tube and a device for collecting blood into said blood collection tube. The device includes: a first slot for securing a blood collection tube therein, and a set of gears arranged at an end of the first slot for rotating the blood collection tube; a vacuum pump; a second slot for securing a needle therein; a plurality of conduits, each conduit connected at a distal end thereof to a reservoir; and a processor configured to specify a predetermined volume of additive to be delivered into a blood collection tube from one of the plurality of reservoirs and a predetermined volume of blood to be delivered into the blood collection tube via the needle. The vacuum pump, a plurality of conduits, and a second slot are arranged around the first slot such that when a blood collection tube having a vacuum port, first septum, and second septum is inserted into the first slot, the vacuum pump is configured to draw a vacuum through the vacuum port; each conduit is fluidically connectable to an interior of the blood collection tube via the second septum; and a needle secured within the second slot is fluidically connectable to an interior of the blood collection tube via the first septum. The processor is configured to control a vacuum delivered from the vacuum pump to draw the predetermined volumes of additive and blood into the blood collection tube. Following the drawing of the predetermined volumes of additive and blood into the blood collection tube, the processor is configured to operate the gears to rotate the blood collection tube a plurality of rotations within the first slot, thereby mixing the additive and blood within the blood collection tube.

Optionally, the device further includes a laser etching tool situated parallel to the axial extent of the blood collection tube and, wherein, during rotation of the blood collection tube, the laser etching tool is configured to etch patient information onto the axial extent of the blood collection tube.

Optionally, the blood collection tube further includes a coating of sizing in a region onto which the laser etching is applied.

Optionally, the device further comprises a gyroscope for measuring orientation, and the processor is configured to permit the operation of the vacuum only when the device's orientation is within a prescribed range.

Optionally, the device further includes an ultraviolet light for internal sterilization.

Optionally, the device further includes an empty blood tube magazine and a full blood tube storage compartment and means for transferring an empty blood collection tube from the magazine to the first slot and for transferring a filled blood collection tube from the first slot to the storage compartment.

According to a second aspect, a method of collecting blood into a blood collection tube is disclosed. The blood collection tube comprises a tube body including a proximal end and a distal end, and defining an axial extent between the proximal end and the distal end; a chamber within the tube body; mixing ribs within the chamber; a vacuum port; and a cap arranged on the proximal end, wherein the cap comprises: a top face including a first septum; a first conduit having an inlet extending from the first septum and an outlet in fluid communication with the chamber; mixing ribs in fluid communication with the chamber, a lateral face including a second septum; and a second conduit extending radially from the second septum to the first conduit. The method comprises: drawing, with a vacuum, a volume of blood additive into the blood collection tube via the second septum through the second conduit; and drawing, with a vacuum, a volume of blood into the blood collection tube via the first septum through the first conduit; and rotating the blood collection tube a plurality of times along an axial extent thereof, to thereby swirl the blood and additive around the mixing ribs of the cap and of the chamber and thereby mix the blood and additive.

Optionally, the rotating step comprises rotating the blood collection tube at least ten times.

Optionally, the method further includes etching patient information on an axial extent of the tube with a laser etching device.

### BRIEF DESCRIPTION OF THE DRAWINGS

FIG. 1A is an isometric view of a blood collection tube with the cap on, according to embodiments of the present disclosure;

FIG. 1B is a cross-section view of the blood collection tube in FIG. 1A with the cap on, according to embodiments of the present disclosure;

FIG. 2A illustrates a bottom view of the cap, showing the mixing ribs therein, according to embodiments of the present disclosure;

FIG. 2B illustrates a top view of the blood collection tube, illustrating the mixing ribs therein, according to embodiments of the present disclosure;

FIG. 3 is an exploded view of the cap, showing a polyolefin outer layer and a molded inner layer, according to embodiments of the present disclosure;

FIG. 4A illustrates a path of insertion of a blood additive into the blood collection tube of FIG. 1B, according to embodiments of the present disclosure;

FIG. 4B illustrates a path of insertion of blood into the blood collection tube of FIG. 1B, according to embodiments of the present disclosure;

FIG. 5A is a cross-section view of a vacuum pump tube applied to a vacuum port of the blood collection tube, according to embodiments of the present disclosure;

FIG. 5B illustrates the operation of a valve and a moisture non-permeable membrane at the vacuum port for sequestering blood from the vacuum port, according to embodiments of the present disclosure;

FIGS. 6A-6C illustrate the filling of a prior art blood collection tube and the blood collection tube of FIGS. 1A-1B filled with blood, according to embodiments of the present disclosure;

FIG. 7A is an upper perspective view of a blood collection device, according to embodiments of the present disclosure;

FIG. 7B is an upper perspective view of the blood collection device of FIG. 7A with a blood collection tube and a blood needle contained therein, according to embodiments of the present disclosure;

FIG. 7C is a lower perspective view of the blood collection device of FIG. 7A, according to embodiments of the present disclosure;

FIG. 7D is an upper perspective view of the blood collection device of FIG. 7A with its casing and certain internal components removed, according to embodiments of the present disclosure;

FIG. 7E is a lower perspective view of the blood collection device of FIG. 7A with its cover and certain internal components removed, according to embodiments of the present disclosure;

FIG. 8A is a depiction of a second embodiment of a blood collection device, including gears for rotation of the blood collection tube for mixing, according to embodiments of the present disclosure;

FIG. 8B illustrates the mixing of blood and additive through rotation of the blood collection tube, according to embodiments of the present disclosure;

FIG. 9 is a perspective view of the vacuum pump of the blood collection device connecting with a vacuum port on the blood collection tube, according to embodiments of the present disclosure;

FIG. 10 is a perspective view of a battery and a corresponding battery slot of the blood collection device, according to embodiments of the present disclosure;

FIG. 11A is a lower perspective view of a system for delivering blood additives from a plurality of liquid reservoirs to a septum of the blood collection tube, according to embodiments of the present disclosure;

FIG. 11B is an illustration of a needle from a blood additive reservoir injecting a blood additive into a blood collection tube, according to embodiments of the present disclosure;

FIG. 12 depicts steps of a method of collecting blood according to embodiments of the present disclosure;

### DETAILED DESCRIPTION OF THE INVENTION

The present Application relates to the collection of blood for testing and, more specifically, but not exclusively, to a blood collection tube, blood collection device, and method for collecting volumes of blood into collection tubes that are stored at atmospheric pressure.

Before explaining at least one embodiment in detail, it is to be understood that the invention is not necessarily limited in its application to the details of construction and the arrangement of components and/or methods set forth in the following description and/or illustrated in the drawings. The invention is capable of other embodiments or of being practiced or carried out in various ways.

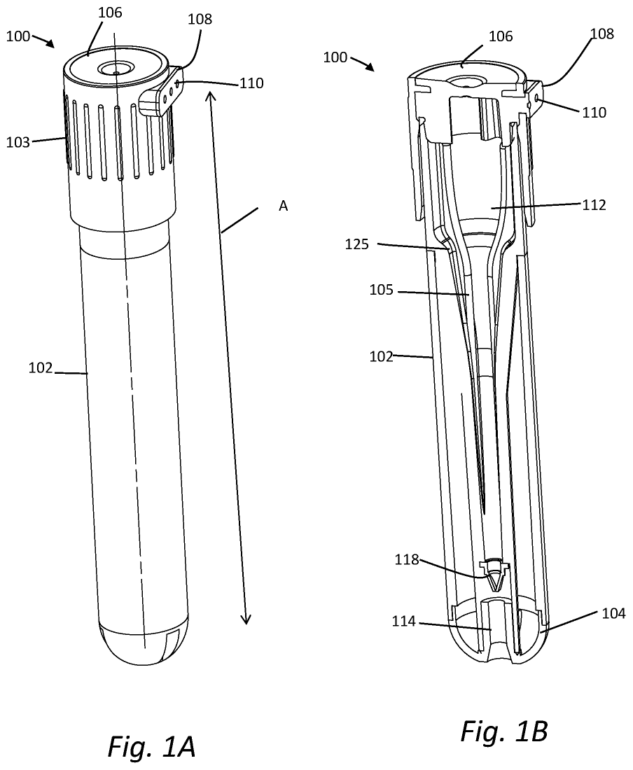

FIGS. 1A and 1B illustrate blood collection tube 100, according to embodiments of the present disclosure. FIG. 1A is an isometric view of the blood collection tube and FIG. 1B is a cross-section view of the tube of FIG. 1A, cut along axis “A.” Blood collection tube 100 includes body 102, defining a chamber therein, and a cap 103. Body 102 defines a proximal end, at cap 103, a distal end, at the bottom of the body 102, and an axial extent therebetween.

In preferred embodiments, the outer dimensions of body 102 and cap 103 are geometrically identical to those of conventional blood collection tubes. For example, the axial length of tube 100 may be 75 mm, and the diameter of the cap 103 may be 13 mm. Advantageously, tube 100 is accordingly compatible with existing equipment for storing blood collection tubes and sampling blood collected in blood collection tubes. In addition, a technician using tube 100 will be comfortable with its shape and size.

Body 102 includes chamber 112 having a funnel-shaped geometry, which defines the contours of chamber 112. Chamber 112 includes a substantially conical upper portion and a substantially cylindrical lower portion with a narrower diameter than the upper portion. The funnel-shaped chamber 112 is used to make the blood height within the chamber compatible with the height of blood in chambers of conventional blood collection tubes. The conventional tubes collect far more blood than necessary and thus have larger volumes than chamber 112. Tube 100 is designed to collect volumes of between 300 μl and 1 ml, whereas conventional tubes are designed to collect between 3 and 5 ml. Without a funnel-shaped geometry, blood collected in tube 100 would pool at the very bottom of body 102, causing difficulty in accessing the collected blood for analysis. For this reason, funnel- shaped chamber 112 limits the effective volume of the blood collection tube 100 and also ensures that the collected blood is accessible at an equivalent height compared to blood collected in a conventional tube. The body further includes a relief channel 125. The relief channel 125 is an indentation along the axis of the tube that allows air to escape as blood enters the tube. This enables uniform blood flow within the tube and prevents the occurrence of an “air jam.”

While the structure of tube 100 is particularly advantageous for the collection of micro-volumes of blood, there is no technical impediment in the use of tube 100 for the collection of larger volumes. For example, the collected blood volume may be as high as 10 ml, as in currently used vacuum blood collection tubes. The shape of chamber 112 may be adjusted as needed to define a desired volume.

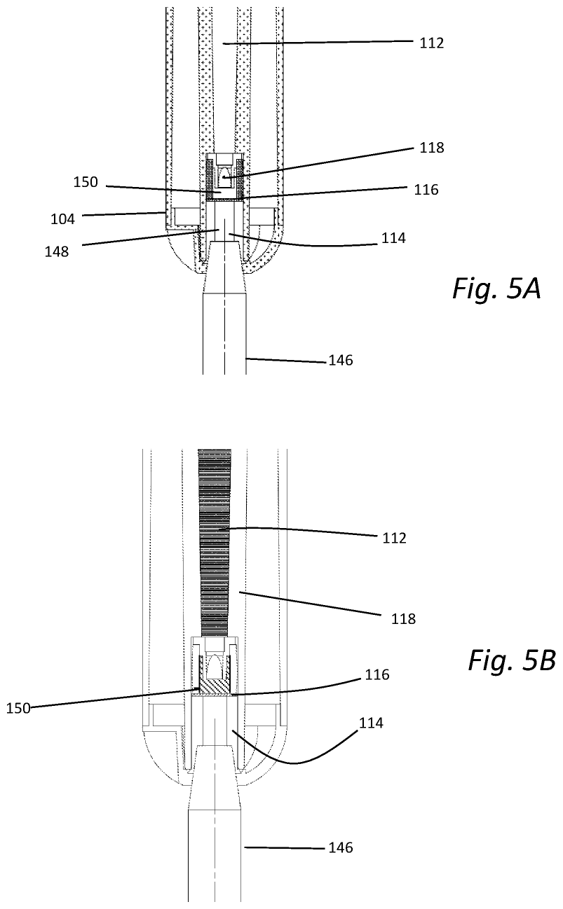

At a distal end of body 102 (i.e., at an end further from cap 103), tube 100 includes a vacuum port 114. Vacuum port 114 is a substantially cylindrical opening configured to interface with tubing from a vacuum pump to draw a vacuum through chamber 112. In the illustrated embodiment, port 114 is at the distal end of body 102; in alternative embodiments, vacuum port 114 is configured along the axial extent of body 102. Between the vacuum port 114 and chamber 112 are liquid-impervious membrane 116 (shown in FIG. 5B) and valve 118, the functions of which will be described further herein.

Optionally, a rupture disk is placed within chamber 112. The rupture disk may be situated toward the top of the chamber and may include a peripheral pathway for the transfer of the blood and the additive therethrough, as well as a central area through which a needle may be inserted during the blood collection. In the illustrated embodiments, no rupture disk is employed, and the blood is retained.

Cap 103 is of a standard size and shape. As seen in FIG. 3, cap 103 consists of an outer shell 109 with an over-molded or inserted septum 106 at a top face. The shell may be made of a polyolefin, while the septum 106 is made of an elastomeric material. This elastomeric material serves as a septum, and a self-sealing gasket, keeping the collected blood and additives secure within the tube until it is desired to remove the blood from the tube. Septum 106 is configured to receive a needle carrying blood from a patient, as well as receive a probe for removing the mixed blood and additive from the tube. Cap 103 further includes septum 108 at a lateral face for receiving an injection of an aliquot of blood additive. Septum 108 optionally includes several defined entry regions 110. Entry regions 110 are locations where the blood additive is inserted into the septum 108. In certain embodiments, entry regions 110 extend through the entire thickness of septum 108, and blood additives are drawn through entry regions 110 by a force of vacuum. In alternative embodiments, entry regions 110 are approximate locations where a needle may be inserted through septum 108, and the additives are inserted via a needle that is extended through the entire thickness of septum 108. The entry regions 110 may be separate, as illustrated, or may be a single large orifice suitable for receipt therein of a multi-lumen needle.

Septum 106 is significantly thinner than an equivalent prior-art septum. Because tube 100 is not stored under vacuum, a thick septum is not necessary to maintain a pressure differential within the chamber.

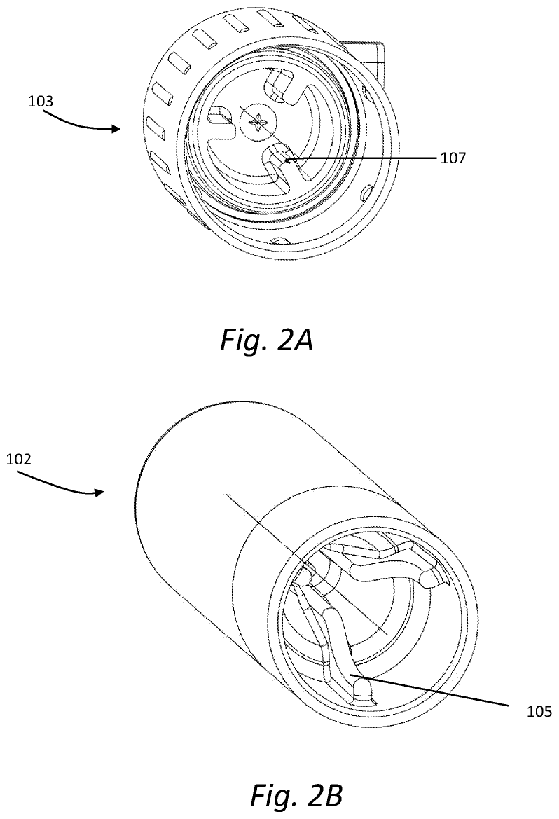

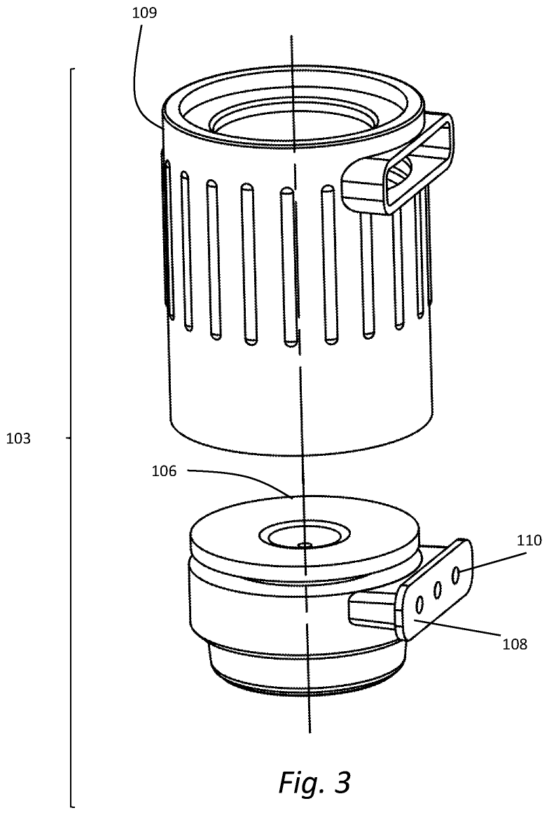

In preferred embodiments, body 102 further includes mixing ribs 107 at a proximal end thereof, as seen in FIG. 1B and FIG. 2B. The cap 103 may further include optional mixing ribs 105, as seen in FIG. 2A. The mixing ribs may be formed integrally with the septum of the same elastomeric material. Mixing ribs 105 and 107 promote swirling of liquid within the cap 103 and chamber 102 when the tube is rotated around its axial extent. This swirling ensures that the additive and blood collected into tube 100 are mixed effectively. Although, in the illustrated embodiments, three mixing ribs are used, more or fewer mixing ribs may also be employed, as desired.

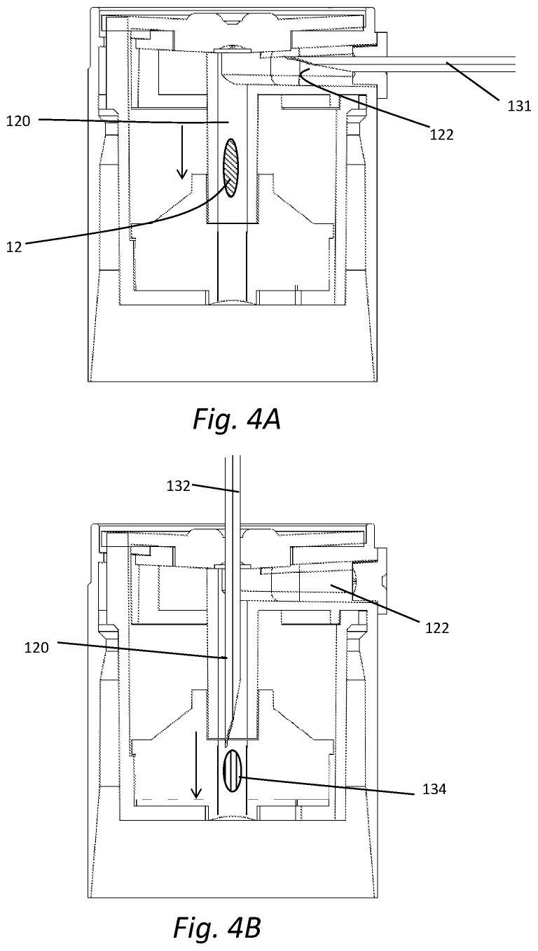

Referring to FIGS. 4A and 4B, insertion of additive and blood into tube 100 proceeds in two stages. As shown in FIG. 4A, a needle 131 is inserted through the septum, and the additive is deposited in the chamber through the second conduit 122. In preferred embodiments, this needle is a tri-lumen needle. Each lumen is connected to a reservoir for one particular additive, and that additive is pumped through its lumen into the septum. Referring to FIG. 4B, after insertion of the additive, blood is injected through septum 106 and into first conduit 120 via needle 132.

After the introduction of the additive and the blood, the blood and the additive are mixed. In a preferred embodiment, the blood and additive are mixed in an active mixing process through rotation of tube 100 around the axis A-A thereof. To effect this mixing, the tube is rotated through the operation of gears within the blood collection device, which will be described further herein. The mixing process causes the blood and additive to mix a suitable amount of times, such as ten times. The mixing ribs promote swirling within the tube. In conventional blood collection systems, the phlebotomist manually inverts a blood collection tube up to ten times to mix the blood and the additive. The combination of the mixing ribs with rotational action replaces this manual mixing with an automatic mixing process.

In the illustrated embodiment, three mixing ribs are located in tube 100, in addition to the three mixing ribs in cap 103. This number of mixing ribs is merely exemplary, and a greater or lesser number of mixing ribs may also be employed.

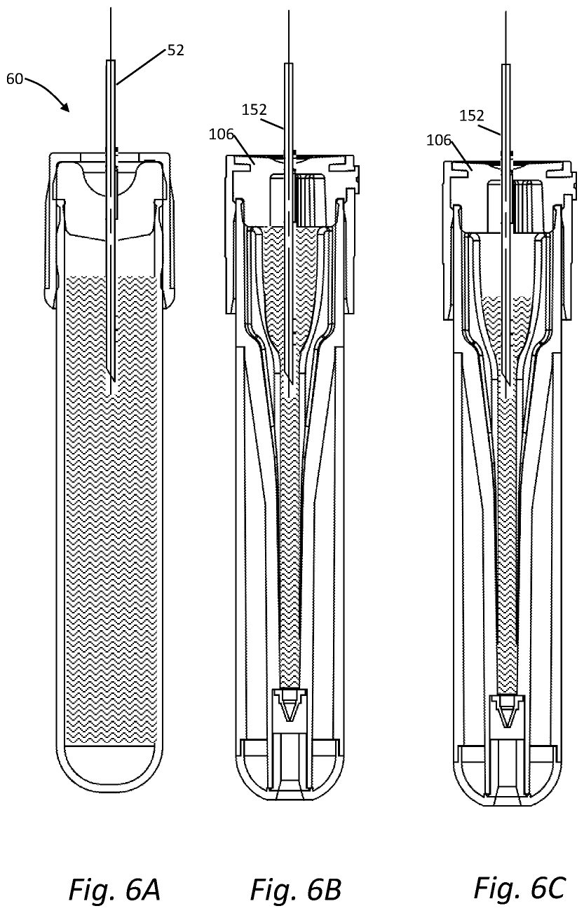

FIGS. 5A and 5B illustrate the insertion of vacuum tube 146 into an interior region 148 of vacuum port 114 and various components of the distal end of tube 100. Membrane 116 is configured between vacuum port 114 and chamber 112. Membrane 116 is configured to permit vacuum to be drawn therethrough but is non-permeable to liquid. For example, membrane 116 may be made of polytetrafluoroethylene (PTFE) or a thin silicone membrane. One-way valve 118 is configured between a distal end of chamber 112 and membrane 116. Valve 118 is made of a material approved by regulatory agencies for contacting blood samples, such as silicone. Valve 118 is controlled by the force of a vacuum applied at port 114. Region 150 is defined between valve 118 and membrane 116. When a vacuum is applied to vacuum port 114, blood is drawn through valve 118 and fills region 150, as shown in FIG. 6B. This blood is sequestered from the distal end of the chamber by the valve. Advantageously, sequestering the blood in region 150 from the rest of the blood in the chamber prevents any contamination of the blood by the membrane 116, which could affect the accuracy of an assay of the blood.

FIGS. 6A-6C illustrate a process of blood withdrawal from the chamber, and illustrate how the same tools may be used to withdraw blood from the chamber of tube 100 compared to conventional tubes. In FIG. 6A, a conventional tube 60 is illustrated. The tube is filled with blood, and a probe 52 is inserted to withdraw the blood. In FIGS. 6B and 6C, blood collection tube 100 is filled with blood. A probe 152 is inserted through septum 106 and accesses the blood within chamber 112. In FIG. 6C, only 300 μl of blood is collected in chamber 112. Chamber 112 is shaped such that the cylindrical-shaped lower section holds less than the collected 300 μl, and accordingly, at least a portion of the collected blood is in the cone-shaped upper section of chamber 112. Because of the smaller volume of chamber 112 compared to that of the conventional tube, the height of the blood is similar, even though much less blood is collected. As a result, probe 152 is easily able to collect blood from the upper section of chamber 112 without having to be longer or thinner than probe 52.

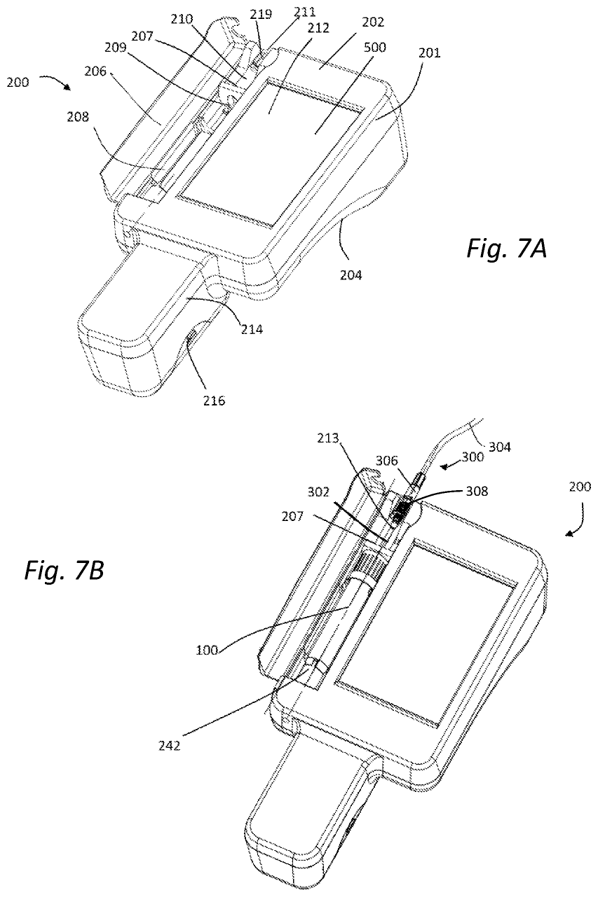

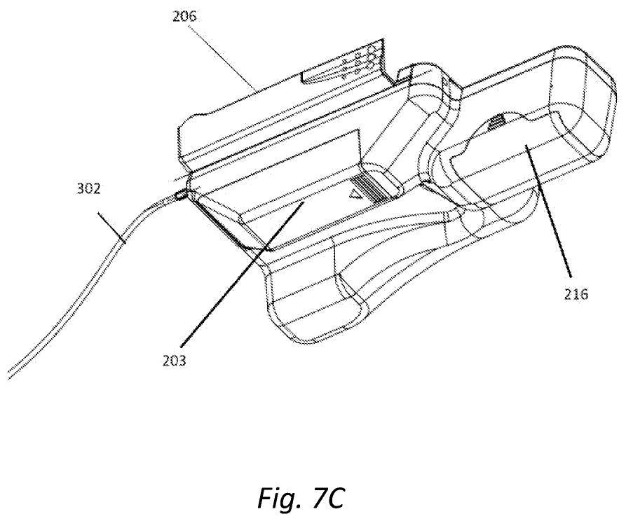

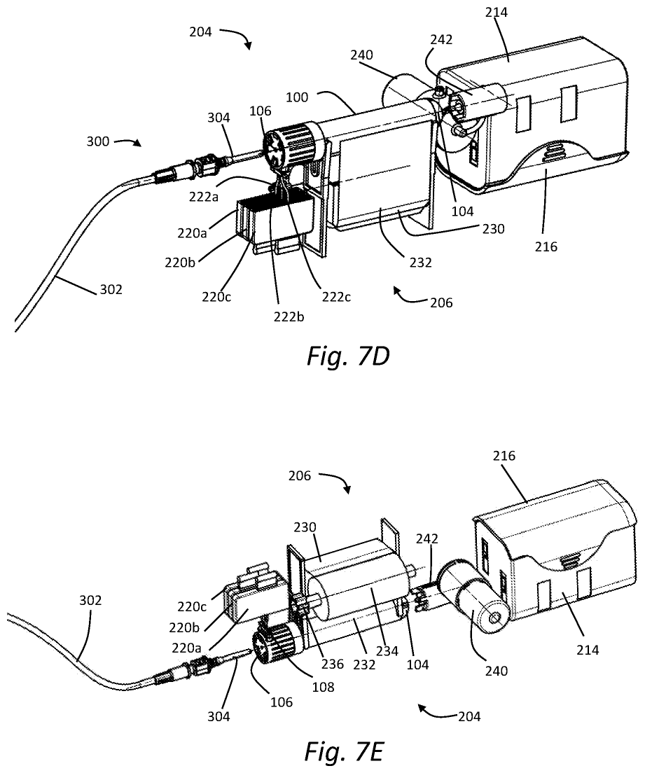

Referring now to FIGS. 7A-11B, blood collection device 200 is configured to withdraw blood and additives into blood collection tube 100. FIG. 7A depicts an upper perspective view of device 200 without a tube therein and FIG. 7B depicts the same view of device 200, with tube 100 therein. FIG. 7C depicts a lower perspective view of device 200.

Device 200 includes casing 201 and has an upper face 202 and a lower face 204. Casing 201 is ergonomically shaped for multiple grip positions for both right-handed and left-handed users.

Cover 206 is attached to casing 201, for example, via a hinged connection. Cover 206 covers the first slot 208 and a second slot 210. Cover 206 may be opened to permit the insertion or removal of tube 100 and is closed and optionally locked during the operation of device 200.

First slot 208 is configured for receiving therein tube 200, and second slot 210 is arranged for receiving therein needle 304. Needle 304 is part of a phlebotomy assembly 300, including a venipuncture cannula (not shown), tubing 302 extending from the venipuncture cannula, needle 304, and connector 306 for connecting the needle 304 with tubing 302. A divider 207 is configured between the first slot 208 and the second slot 210. The divider 207 has a central aperture 209 for permitting needle 304 to pass therethrough. Similarly, cover 206 has an aperture 211 parallel to aperture 209, for permitting the needle 304 to pass therethrough, even when the cover 206 is closed. An optical sensor (not shown) may be arranged at divider 207 or central aperture 209. The optical sensor may be used to confirm whether blood has entered needle 304, for example, during a priming process of tubing 302 and needle 304.

Optionally, the needle 304 includes a blood metering sensor 308. The blood metering sensor measures a volume of blood that passes through the needle 304. This is especially relevant during a priming process, as the connector 306 is initially filled with air, and the air needs to be evacuated before a volume of blood may be drawn with accuracy. The blood metering sensor measures the blood passing through the needle, thereby determining that the blood volume matches the expected volume. The blood metering sensor may perform these sensations through any specific measuring technique known to those of skill in the art, such as measuring sound or light.

Second slot 210 further includes a locking slot 219. Locking slot 219 is shaped to receive connector 306 and needle 304 and is slidable within the second slot 210. The second slot 210 may also have a locking tube 213 for receiving the needle 304 therein. When the needle 304 is received in locking slot 219 and locking tube 213, and the needle 304 is advanced relative to the second slot 210, the locking slot 219 and locking tube 213 advance with it. The advancing locking tube 213 pushes tube 100 within the first slot 208. Tube 100 is pushed sufficiently forward by locking tube 213 to be flush with vacuum adapter 242. In alternative embodiments without a locking tube 213, a similar pushing function may be performed by the needle 304 or connector 306 itself. The locking slot 219 may be locked into place with a spring-loaded mechanism attached to a sensor (not shown).

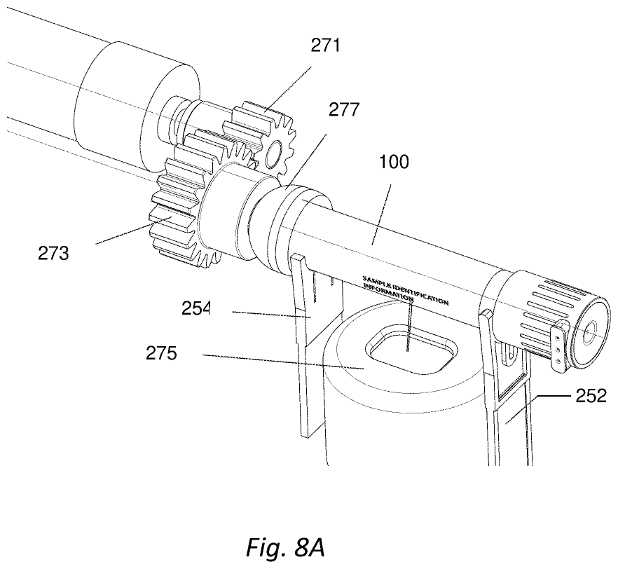

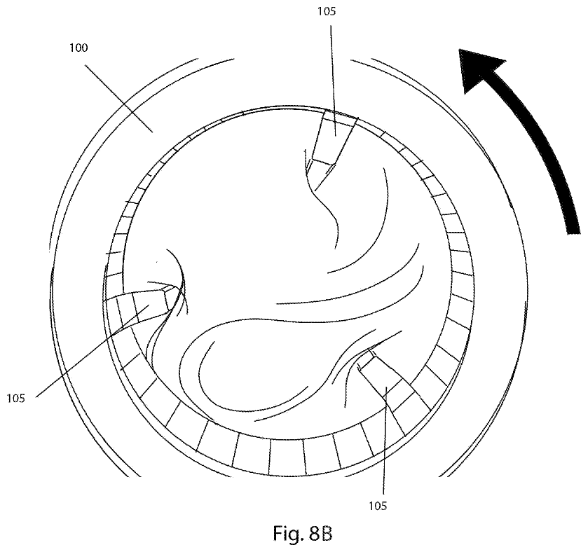

Optionally, locking slot 219 and needle 304 may be advanced, locked, and retracted in an automated fashion between three positions: a rest position, in which the tube 100 is not flush with vacuum adapter 242; a vacuum position, in which the tube 100 is flush with vacuum adapter 242 but the needle 302 is not piercing septum 106; and a drawing position, in which the needle 302 is advanced so that it is piercing septum 106. The locking slot 219 may be controlled by any suitable motor, such as a servo motor. The needle 304, locking slot 219, and tube 100 stay in place until completion of a blood collection process, at which point the locking slot 219 is automatically released, causing automatic retraction of locking slot 219 and ejection of the needle 304 from tube 100. The view of FIG. 8B shows tube 100, locking slot 219, and locking tube 213 in this released position. The foregoing descriptions of locking slot 219 and locking tube 213 are merely exemplary, and any other suitable mechanism may be employed for releasably locking the needle 304 and tube 100 in place.

Screen 212 is also visible within upper face 202. Screen 212 may be a touch screen. The touch screen 212 may be any standard screen or display suitable for implementation in a mobile computing device, such as LCD, OLED, AMOLED, Super AMOLED, TFT, or IPS. The screen displays a graphic user interface 500 for the operation of device 200.

Screen 212 is integrated with a processor (not shown) for controlling the operations of device 200. The processor includes memory and circuitry for executing computer-readable program instructions stored in the memory. The memory is a non-transitory storage medium having stored thereon code instructions that, when executed by the processor, cause the performance of various steps. The storage medium may be, for example, an electronic storage device, a magnetic storage device, an optical storage device, a semiconductor storage device, or any suitable combination of the foregoing. In particular, the functions described herein may be programmed by a computer program product installed on the non-transitory computer-readable medium of the processor. In exemplary embodiments, the screen 212 functions as an input interface for the processor, including for confirming predetermined volumes of blood additive and blood to be delivered into tube 100.

In addition, the processor preferably includes wireless communication hardware, such as Wi-Fi or Bluetooth, for transmitting information between the device 200 and an external device, such as a facility computer system, a smartphone, or a tablet. Alternatively or in addition, device 200 includes a manual data port, such as a USB connection, for interfacing between the processor and the external device. Through the link to the facility system, the processor is used to control patient information, sample collection compliance, specimen quality optimization, documentation compliance, and to provide complete inventory and storage management. The data link may also be used to provide programming, maintenance, and software updates to device 200. A computer program product for controlling and documenting patient blood draws may be installed on both the facility computer system and on device 200, for ease of compatibility and information transfer.

Optionally, the screen 212 and processor are part of a tablet computer that is installable within and removable from device 200. Alternatively, the screen 212 and processor may be permanently fixed within device 200.

The processor is connected to various sensors in device 200, including a sensor for determining whether cover 206 is open or closed, a sensor for determining whether locking tube 213 is in a locked or unlocked position, a gyroscope sensor for determining if the orientation level is acceptable, and other sensors that will be described further herein.

Device 200 further includes battery 214, which is stored within battery case 215 (shown in FIG. 10). Battery case cover 216 secures battery 214 within battery case 215. Battery 214 may be any battery known to those skilled in the art, such as lead-acid, nickel cadmium, nickel-metal hydride, lithium-ion, lithium-ion polymer, or an alkaline battery. In advantageous embodiments, battery 214 is rechargeable. Because device 200 is battery-powered, device 200 is fully portable. Battery 214 is configured to last throughout a typical user's workday.

In addition to being portable, in preferred embodiments, device 200 is handheld. That is, the device 200 can be held by a technician during use. In further advantageous embodiments, the device 200 may be held with a single hand, so a technician may use one hand to hold the device 200 and another to insert or remove a needle or collection tube. In preferred embodiments, the size of device 200 is comparable to that of handheld printers currently known to those of skill in the art, for example, Zebra® printers.

FIGS. 7D and 7E depict device 200 with casing 201 and certain other internal components removed. In the view of FIG. 7D, top side 204 is on the top, and bottom side 206 is on the bottom; in the view of FIG. 7E, these orientations are reversed.

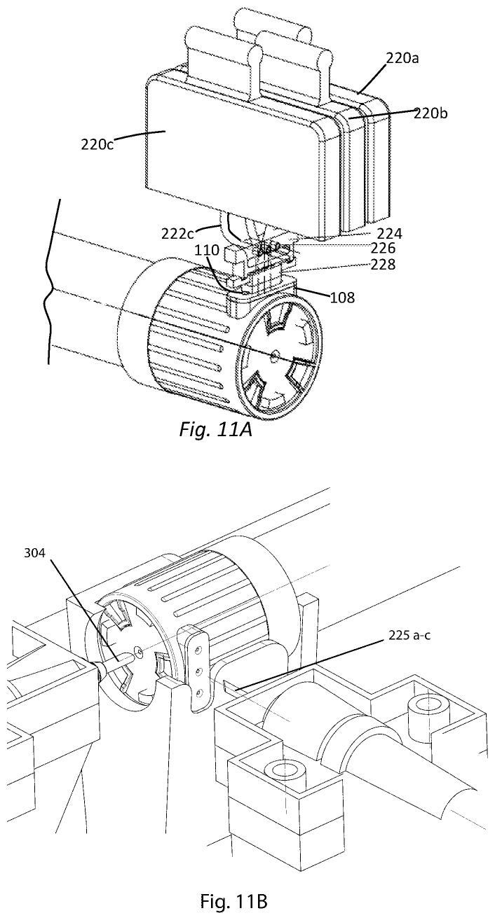

Reservoirs 220a, 220b, and 220c store blood additives. Although in most examples described below, reservoirs 220 contain liquid additives, the term “reservoir” encompasses a store of a solid additive. Typically, each reservoir 220a-c stores a different additive. For example, reservoir 220a may store a concentrated solution of sodium heparin or lithium heparin; reservoir 220b may store a concentrated solution of potassium EDTA, and reservoir 220c may store a concentrated sodium citrate solution. The number of reservoirs and the type of additive in each reservoir may be modified without departing from the scope of the present disclosure. A plurality of conduits 222a, 222b, and 222c are respectively connected to a corresponding reservoir 220a, 220b, 220c. Each conduit 222 extends from a respective reservoir 220 toward septum 108 of tube 100. The specific mechanism for delivery of a blood additive from reservoir 220 through conduit 222 will be described further in connection with FIGS. 11A-11B.

Also visible in FIGS. 7D and 7E is printer 230, whose location is indicated schematically. Printer 230 may be, for example, a thermal printer or a color micro printer. A roll of adhesive-backed labels 234 is arranged adjacent to printer 230. A single label 232 extends upward from roll 234 toward tube 100. Roller 236 is used to advance roll 234 as the printer 230 is printing.

FIG. 8A illustrates another embodiment of device 200 with casing 201 and certain other internal components removed. In this embodiment, the printer is not present, and instead, a laser etching device 275, whose location is schematically indicated, is used to imprint patient information directly on the tubes, as discussed. In addition, gears 271 and 273 are configured to rotate the tube 100 following insertion of the additive and blood to mix the additive and the blood, as discussed. Suction fitment 275 secures the end of tube 100 during rotation thereof. Although, in the illustrated embodiments, the tube rotation is described as along the long A-A axis and is performed through the operation of gears 271 and 273, other methods of implementation are possible for the mixing process. For example, the mixing may take place through vibration or anti-axial methods (i.e., back-and-forth movement in a perpendicular direction to the axial extent).

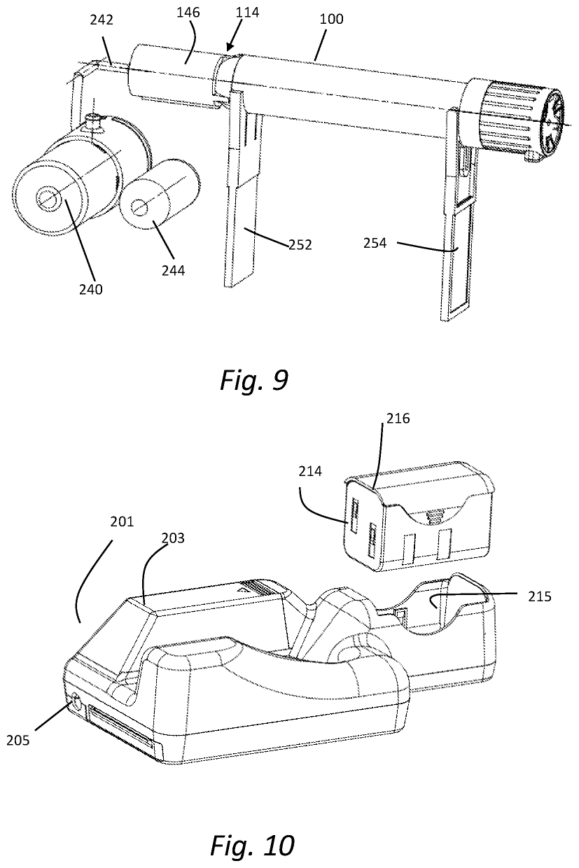

Referring to FIG. 9, device 200 further includes vacuum pump 240. Vacuum pump 240 may be a micro-diaphragm gas sampling pump. Vacuum pump 240 includes adapter 242 for receiving the bottom of tube 100 and making a vacuum-tight seal therewith. The interaction between pump 240 and tube 200 is further shown in FIG. 9. The tube 100 rests on stands 252 and 254, with the bottom 104 at a rear portion of the device 200. Vacuum tube 146 extends from adapter 242 and is insertable into the vacuum port 114 of tube 100 in the manner discussed above. The specific vacuum drawn by the vacuum pump through vacuum tube 146 may be controlled by the processor, for example, through the use of sequencing valves.

Optionally, vacuum pump 240 is connected to a vacuum reservoir or staging chamber 244 to store a vacuum. The stored vacuum may be used to enable a quicker or more controlled vacuum application to tube 100.

Referring to FIG. 10, device 200 further includes a barcode scanner 205, whose location is schematically indicated within casing 201. Barcode scanner 205 is used to scan a barcode on a patient's armband to verify the patient's identity. Other sensors may alternatively be used to confirm patient identity, including, for example, image sensors, facial recognition technology, or digital capture technology. Device 200 also includes a removable cover 203 for inserting and removing reservoirs 220a-220c, as well as for inserting and removing ink cartridges for printer 230.

Other optional components of the device 200 include an internal ultraviolet light for sterilization of the interior of the device 200 in between patients, a gyroscope for determining the orientation level of the device, and for enabling the use of the device only when the device is at a proper orientation for optimal performance; a tube magazine, for enabling the possibility of collection of multiple tubes without operator intervention, a storage area for the completed and labeled tubes, and means for transporting the empty and full tubes before and after blood collection (e.g., pick and place arms, conveyor belts, or magnetic grippers).

FIGS. 11A-11B illustrate in greater detail the system for delivering blood additives from the reservoirs 220a-220c, through conduits 222a-222c, and into septum 108 of tube 100, according to embodiments of the present disclosure. As shown in FIG. 11A, valve assembly 224 is configured between each respective reservoir and an outlet of each conduit 222a, 222b, 222c. Valve assembly 224 contains, for example, a plurality of solenoid valves, each configured to control fluid flow through a respective conduit 222. The system further includes an air intake 226 and a sensor 228 configured to monitor fluid flow through each valve in valve assembly 224. Sensor 228 may be, for example, any type of flow meter known to those of skill in the art. As shown in FIG. 11B, each conduit 222a-222c terminates in a needle 225a-225c. Each needle 225a-225c is operatively connected to a motor, such as a servo motor having a position encoder configured to extend and retract the needle. The needles 225a-225c may alternatively be part of an integrated multi-lumen needle, as discussed, in which case a single servo motor operates the multi-lumen needle.

A process of drawing a blood additive from a reservoir 220 into a tube 100 using vacuum pump 240 is as follows. First, an identity and volume of blood additive to be drawn is set with the processor. Upon determination of a volume of fluid to be delivered from a particular reservoir 220, the processor directs the operation of the servo motor so that the needle 225 corresponding to that reservoir 220 extends and penetrates the septum 108, as shown in FIG. 11B. The processor further directs the opening of a valve within valve assembly 224 corresponding to the selected additive. The opening of the valve permits an additive to be drawn out of the selected reservoir 220. A vacuum is applied by vacuum pump 240, drawing a vacuum through tube 100, including through first conduit 120 and second conduit 122, through needle 225, and through conduit 222. As a result, the additive is drawn from reservoir 220. When sensor 228 determines that the predetermined volume of the additive has passed through valve 224, the processor directs the closure of the valve and opens air intake 226. Air intake 226 delivers a small pocket of air behind the aliquot of concentrated blood additive to help drive the aliquot through conduits 222, needles 225, and first and second conduits 120 and 122 of the tube. The vacuum is then turned off, and the needle 225 is removed from septum 108.

Alternative additive delivery methods from reservoir 220 include piezo or inkjet delivery.

In the above-described embodiments, the blood additives are provided as highly concentrated liquid solutions. The concentration is determined such that a quantity of up to around a 10 μl aliquot of the solution is sufficient for providing additive to a blood draw of up to 1 ml. For example, lithium heparin may be delivered either at 15.8 USP (United States Pharmacopeia units) per unit or pulled as a 10 μl aliquot from a solution that has an equivalent 1580 USP. EDTA may be provided in a super concentrate that contains 180 mg of EDTA for every 10 uμl aliquot. These concentrations are merely exemplary, and other concentrations may also be employed.

One advantage of using concentrated liquid solutions is that the blood additives are removed from needles 225 without requiring any liquid or outside material to contact the tips of needles 225. As a result, it is possible to use the same delivery system to deliver blood additives to multiple tubes 100 without any risk of contamination. In addition, because there are separate conduits 222 for each blood additive and separate regions 110 on septum 108 for receiving the different additives therethrough, there is no possibility of contamination of any particular reservoir 220 or conduit 222 with a blood additive from a different reservoir or conduit.

In alternative embodiments, the blood additives may be delivered as solids. For example, the blood additives may be delivered with a screw conveyor system, wherein the tip of each screw conveyor is configured as a needle that is insertable into septum 110. The device may also use a combination of liquid and solid delivery as needed, with the liquid and solid additives being delivered from separate reservoirs 220.

In addition, in alternative embodiments, instead of using vacuum pump 240 to withdraw the blood additives, a separate delivery system is implemented for them. For example, the separate delivery system may include a vacuum push-pull system configured at reservoir 220.

In a preferred embodiment, patient information is supplied onto the tube through laser etching. The etched information may include, for example, a patient's name, identification number, barcode, QR code, etc. The tube is rotated through operations of gears, for example (illustrated in FIG. 8A) during the etching process. For the etching to be effective, the tubes are made of a material susceptible to laser etching, such as polycarbonate. Optionally, each tube is coated with a layer of sizing around the etching region to increase the contrast during the laser etching. The sizing may also serve to protect the blood within the tube from the laser etching. Advantageously, compared to printing, the use of laser etching reduces the volume of consumables (such as toner and labels) that are required to be supplied to the device 200 during the operation of the device.

In alternative embodiments, patient information is supplied through label printing, as discussed at length in application WO2022/038616.

While device 200 is depicted in the illustrated embodiments as a standalone device, it is also possible to integrate it into a larger device. For example, a robotic surgery device, whether for general surgery or orthopedic surgery, may include a blood sampling module with all of the operative components of device 200.

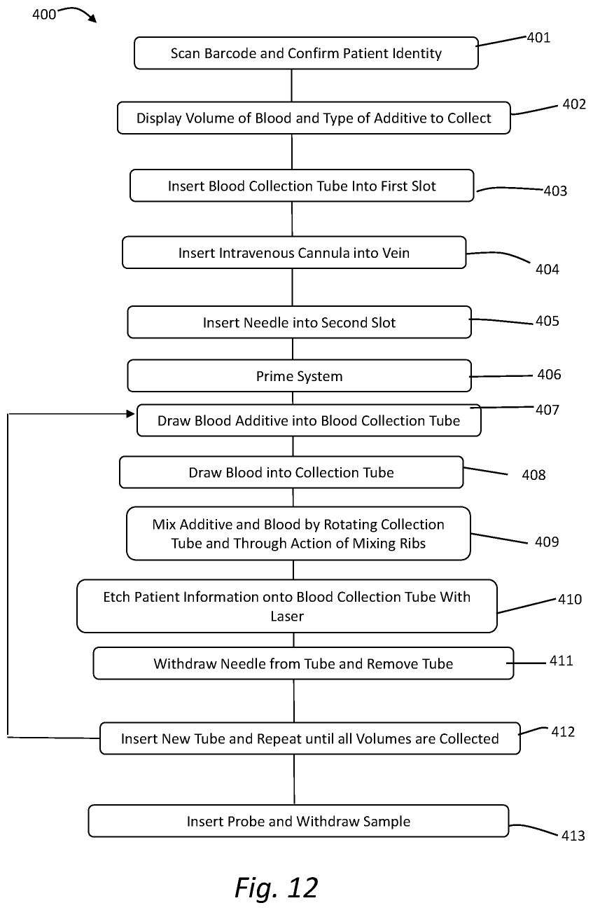

Referring now to FIG. 12, a method 400 of drawing blood into tube 100 with device 200 is disclosed.

At step 401, a user scans a bar code associated with a patient. The bar code may be printed on an armband worn by the patient. The processor determines patient information based on the scanned bar code, for example, the patient's name and current location. The processor compares the patient information to stored patient information previously downloaded to the device 200. For example, the processor may have stored thereon a list of patients and bar code information for which blood drawing is required. The processor may allow proceeding to the next step of blood collection only when the patient's name and scanned bar code information match one of the names and bar codes on the list. This prevents errors due to misidentification.

At step 402, device 200 displays information regarding how much blood to collect from the patient, which blood additives to include in each tube, in which order, and any special instructions associated with that patient, such as drawing at an especially low vacuum. The volumes of blood and additives to be collected are predetermined. For example, a physician may order a list of assays, and the laboratory information system or the processor of device 200 sets the volume of blood required to be collected to perform such assays. The processor or computer program may further calculate a predetermined volume of blood additive necessary to add to each blood sample to preserve the blood for testing.

At step 403, the technician inserts a blood collection tube into first slot 208 and closes cover 206. Because the blood collection tubes 100 are all interchangeable, this step may be performed before any of the preceding steps. Blood collection tube 100 is maintained at atmospheric pressure prior to insertion into first slot 208. As used in the present disclosure, “atmospheric pressure” refers to the ambient pressure of an environment where the blood drawing process is performed.

Optionally, if the door is not closed and locked into place, an error message appears, and the screen does not proceed to the remaining steps. Additionally, there may be an indicator of the acceptable range of level orientation. For example, the indicator may be green if the device is in the acceptable range and red if the device is outside the acceptable range. If the indicator is red, the device needs to be repositioned before proceeding with the collection. The device will not function if the indicator is red.

At step 404, the user inserts an intravenous cannula into a vein. The user may perform this step manually in a manner known to those with skill in the art. For example, the technician may insert a winged needle set into the vein. The cannulation and drawing of blood may alternatively be performed by a phlebotomy robot.

At step 405, the technician inserts the needle 304 into the second slot 211. The technician locks needle 304 into place using locking slot 213 and/or locking tube 219.

At step 406, the user primes the system. The priming may occur automatically or in response to user instruction. The optical sensor determines that the system is primed by sensing the presence of blood in tubing 302 or needle 304.

At step 407, device 200 draws a volume of blood additive solution from the appropriate reservoir into blood collection tube 100 by applying a vacuum at the vacuum port 114. This step may be performed in the manner described above in connection with FIGS. 11A-11B. Specifically, a vacuum is applied to blood collection tube 100 while cap 103 of blood collection tube 100 is fluidically connected to reservoir 220 containing the blood additive.

The volume of blood additive is added to the tube according to a predetermined ratio of additive to blood. The ratio may be, for example, about 1:100. Thus, for a 500 μl sample of blood, 5 uμl of additive is added. It is possible to deviate to some degree from the predetermined ratio without compromising the accuracy of the blood tests.

The vacuum is applied for a particular period of time. Specifically, the vacuum is applied for a sufficient amount of time to withdraw the blood additive into the second conduit 122 and first conduit 120, but not to withdraw the blood additive further into blood collection tube 100. Because a very small volume of blood additive is drawn, it is possible to fit the entire volume of drawn blood additive in the second conduit 122 or first conduit 120.

At step 408, device 200 draws a volume of blood from needle 304 into blood collection tube 100. This drawing step is thus performed by applying a vacuum to the blood collection tube at vacuum port 114, while cap 103 of the blood collection tube is fluidically connected to an intravenous cannula. One advantage of inserting the blood only after insertion of the additives is that there is no potential for contaminating the remaining additives in reservoirs 220 and conduits 222 with blood.

In exemplary embodiments, the applied vacuum may be approximately 120-150 mm Hg. The specific applied vacuum may be selected based on various factors, such as the desired time required for blood collection. The volume of withdrawn blood may be, for example, between 300 and 1,000 μl or more pending usage. The vacuum is applied until the predetermined volume of blood is collected, at which point the vacuum ceases automatically.

Typically, steps 407 and 408 are performed in very close proximity, i.e., within a few seconds of each other. Theoretically, steps 407 and 408 can happen simultaneously, so long as the needle delivering the additive is not contaminated by blood. This may be ensured through various mechanisms, including controlling the insertion depth of the needle containing the additive and the needle containing the blood.

The drawing of the additive and blood proceeds automatically once the user initiates the priming process. Throughout the collection process, a technician may be able to execute an emergency stop to abort the collection. The technician may also control the amount of vacuum applied if he or she determines this is necessary. This vacuum adjustment is recorded by the processor and associated with the patient's electronic medical record.

At step 409, device 200 mixes the blood and additive together. This mixing is performed through the rotation of the tube within the device 200 and through the operation of the mixing ribs. The tube is rotated a number of times, which forces the blood to be mixed with the additive. As illustrated in FIG. 8B, the rotational motion makes the blood fold over itself, utilizing the internal ribs/fins incorporated in the chamber. The mixing step preferably includes mixing the blood and additives at least ten times. Advantageously, this degree of mixing corresponds to a degree of mixing that would be performed manually by a user on a prior art tube.

At step 410, device 200 etches patient information onto the blood collection tube 100 using a laser etching tool. As discussed, the patient information may include the patient name and ID, as well as the additive type, the date, the time, and the technician ID. The laser etching may be performed substantially at the same time as the mixing by applying the laser etching during the rotation of the tube for mixing. Alternatively, instead of laser etching, the patient information may be supplied by printing and affixing a label.

At step 411, the needle 304 is removed from blood collection tube 100. Optionally, this step is performed automatically by releasing a locking mechanism holding the needle and blood collection tube in place, as described above in connection with FIGS. 7A and 7B. The processor uploads information regarding the blood collection to the facility's computer system. This information includes the date and time of collection and the identity of the phlebotomist who collected the sample. A button on the graphic user interface instructs the technician to remove tube 100.

Each of the steps 408-411 are controlled by device 200 with forced compliance and are documented with traceability.

At step 412, optionally, if the patient requires more than one type of blood assay, a new blood tube is inserted, and steps 407-411 are repeated. The graphic user interface may prompt the user to load the next tube, or alternatively, a new tube is conveyed via the tube magazine. Upon sensing the locking of the needle into the locking slot again, the processor may prompt the user to select or confirm values for the predetermined volumes of fluid and blood. In the alternative, the volumes of blood and additive may be set for multiple blood collection tubes prior to the first collection of blood and additive. The process is continued until all required tubes are complete.

When all required tubes are complete, a message may be displayed on the graphic user interface. The message may indicate that collection is complete and instruct the user to remove the needle from the patient and bandage the incision point. Another button may appear, prompting the user to “Upload Collection Information.” When this button is activated, all details of the successful collection are uploaded to the laboratory information system. The patient list is updated with either “complete” (if all tubes are collected) or “partial” (if only some of the requested tubes were collected.

Finally, at step 413, a user withdraws a sample from the blood collection tube for analysis. This user is a lab technician rather than a phlebotomist. Optionally, instead of removing the cap 103 of blood collection tube 100, the user may insert a probe 152 into the cap 103. The probe 152 passes through septum 108 and first conduit 120 to reach the collected blood for sampling, as described above in connection with FIGS. 6B-6C.

What is claimed is:

1. A blood collection tube, comprising:
a body including a proximal end and a distal end, and defining an axial extent between the proximal end and the distal end;
a chamber within the body, the chamber including a plurality of mixing ribs;
a vacuum port; and
a cap arranged on the proximal end, wherein the cap comprises:
a top face including a first septum;
a first conduit having an inlet extending from the first septum and an outlet in fluid communication with the chamber;
a lateral face including a second septum;
a second conduit extending radially from the second septum to the first conduit; and a plurality of mixing ribs;
wherein, when a fluid source is fluidically connected to the first septum or the second septum, and a vacuum is applied at the vacuum port, the vacuum draws fluid from the fluid source, through the first or second septum, and into the first conduit; and
when the tube is rotated around an axis defined by the axial extent, the mixing ribs of the cap and the mixing ribs of the chamber cause fluids therein to swirl, thereby effecting mixing of the fluids therein.

2. The blood collection tube of claim 1, further comprising a relief channel configured to enable air to escape from the chamber during filling of blood into the chamber.

3. A system for collecting blood, comprising the blood collection tube of claim 1, and a device for collecting blood into said blood collection tube, the device comprising:
a first slot for securing a blood collection tube therein, and a set of gears arranged at an end of the first slot, for rotating the blood collection tube;
a vacuum pump;
a second slot for securing a needle therein;
a plurality of conduits, each conduit connected at a distal end thereof to a reservoir;
a processor configured to specify a predetermined volume of additive to be delivered into a blood collection tube from one of the plurality of reservoirs and a predetermined volume of blood to be delivered into the blood collection tube via the needle;
wherein, the vacuum pump, plurality of conduits, and second slot are arranged around the first slot such that when a blood collection tube having a vacuum port, first septum, and second septum is inserted into the first slot:
the vacuum pump is configured to draw a vacuum through the vacuum port;
each conduit is fluidically connectable to an interior of the blood collection tube via the second septum; and
a needle secured within the second slot is fluidically connectable to an interior of the blood collection tube via the first septum;
the processor is configured to control a vacuum delivered from the vacuum pump so as to draw the predetermined volumes of additive and blood into the blood collection tube; and
wherein, following drawing of the predetermined volumes of additive and blood into the blood collection tube, the processor is configured to operate the gears so as to rotate the blood collection tube a plurality of rotations within the first slot, thereby mixing the additive and blood within the blood collection tube.

4. The system of claim 3, wherein the device further comprises a laser etching tool situated parallel to the axial extent of the blood collection tube, and, wherein, during rotation of the blood collection tube, the laser etching tool is configured to etch patient information onto the axial extent of the blood collection tube.

5. The system of claim 4, wherein the blood collection tube further includes a coating of sizing in a region onto which the laser etching is applied.

6. The system of claim 3, wherein the device further comprises a gyroscope for measuring orientation, and the processor is configured to permit operation of the vacuum only when the orientation of the device is within a prescribed range.

7. The system of claim 3, wherein the device further comprises an ultraviolet light for internal sterilization.

8. The system of claim 3, wherein the device further comprises an empty blood tube magazine and a full blood tube storage compartment, and means for transferring an empty blood collection tube from the magazine to the first slot and for transferring a filled blood collection tube from the first slot to the storage compartment.

9. A method of collecting blood into a blood collection tube, wherein the blood collection tube comprises a tube body including a proximal end and a distal end, and defining an axial extent between the proximal end and the distal end; a chamber within the tube body; mixing ribs within the chamber; a vacuum port; and a cap arranged on the proximal end, wherein the cap comprises: a top face including a first septum; a first conduit having an inlet extending from the first septum and an outlet in fluid communication with the chamber; mixing ribs in fluid communication with the chamber, a lateral face including a second septum; and a second conduit extending radially from the second septum to the first conduit; and the method comprises:
drawing, with a vacuum, a volume of blood additive into the blood collection tube via the second septum through the second conduit; and
drawing, with a vacuum, a volume of blood into the blood collection tube via the first septum through the first conduit;
and rotating the blood collection tube a plurality of times along an axial extent thereof, to thereby swirl the blood and additive around the mixing ribs of the cap and of the chamber and thereby mix the blood and additive.

10. The method of claim 9, wherein the rotating step comprises rotating the blood collection tube at least ten times.

11. The method of claim 10, further comprising etching patient information on an axial extent of the tube with a laser etching device.
